(19) United States
(12) Patent Application Publication
(10) Pub. No.: US 2026/0308479 A1
(43) Pub. Date: Oct. 8, 2026

(54) MIXTURE OF LINEAR AND BRANCHED ALKYL POLYGLYCOSIDES

(71) Applicants: Dow Global Technologies LLC, Midland, MI (US); Rohm and Haas Company, Collegeville, PA (US)

(72) Inventors: Daniel S. Miller, Phoenixville, PA (US); Janet McMillan, Bay City, MI (US); Robert D. Kennedy, Midland, MI (US); Matthew M. Yonkey, Sanford, MI (US); Yujing Tan, Midland, MI (US); Jianbo Hou, Midland, MI (US); Zachary Rolfs, Midland, MI (US); Tanner McDaniel, Midland, MI (US); Timothy J. Young, Bay City, MI (US); Thomas Peterson, Midland, MI (US); Dan Ye, Missouri City, TX (US)

(21) Appl. No.: 19/482,030

(22) PCT Filed: May 23, 2024
(86) PCT No.: PCT/US2024/030678

§ 371 Date: Nov. 6, 2025

Related U.S. Application Data
(60) Provisional application No. 63/470,462, filed on Jun. 2, 2023.

(51) Int. Cl. C11D 1/66; C07H 15/04; C11D 3/43

(52) U.S. Cl. CPC C11D 1/66; C07H 15/04; C11D 3/43; C11D 2111/14; C11D 2111/44

(57) ABSTRACT

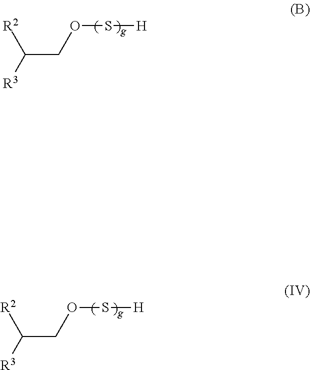

A mixture of alkyl polyglycosides, comprising: a linear alkyl polyglucoside of formula A: R¹—O-(G)ₙH (A) wherein R1 is a linear C6-15 alkyl group; wherein n is an average of 1 to 5; wherein G is a monosaccharide of glucose; and a branched alkyl polysaccharide of formula B (B) wherein R2 is a C2 alkyl group; wherein R3 is a linear or branched C4 alkyl group; wherein g is an average of 1 to 5 and wherein S is a monosaccharide with 5 carbon atoms; with the proviso that R1 is a linear C9-15 alkyl group in at least some of the linear alkylpolyglucoside of formula A; and with the proviso that the mixture of alkyl polyglycosides comprises <3 wt %, based on weight of the mixture of alkyl polyglycosides, of a compound of formula IV (IV) wherein R12 is —(CH2)pCH3; wherein R13 is —(CH2)+1CH(CH3)2; wherein G6 is a monosaccharide with 4 to 6 carbon atoms; wherein f is 1.1 to 10 and wherein p is 1 to 4.

The present invention relates to a mixture of alkyl polyglycosides. In particular, the invention relates to a mixture of alkyl polyglycosides, comprising: a linear alkyl polyglucoside of formula A

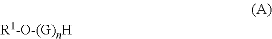

wherein R¹ is a linear C₆₋₁₅ alkyl group; wherein n is an average of 1 to 5; wherein G is a monosaccharide of glucose; and a branched alkyl polysaccharide of formula B

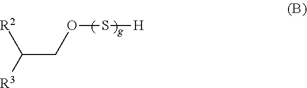

wherein R² is a C₂ alkyl group; wherein R³ is a linear or branched C₄ alkyl group; wherein g is 1 to 5 and wherein S is a monosaccharide with 5 carbon atoms; with the proviso that R¹ is a linear C₉₋₁₅ alkyl group in at least some of the linear alkyl polyglucoside of formula A; and with the proviso that the mixture of alkyl polyglycosides comprises <3 wt %, based on weight of the mixture of alkyl polyglycosides, of a compound of formula IV

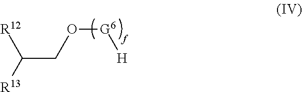

wherein R¹² is —(CH₂)ₚCH₃; wherein R¹³ is —(CH₂)ₚ₊₁CH(CH₃)₂; wherein G⁶ is a monosaccharide with 4 to 6 carbon atoms; wherein f is 1.1 to 10 and wherein p is 1 to 4.

Aqueous cleaning formulations, for example, floor care formulations, hard surface cleaning formulations have a wide array of uses. For example, uses include cleaning hard surfaces such as floors, counters, walls, tables, and other things made of, for example, wood, stone, laminate, ceramic and plastic materials which need to be cleaned periodically of accumulated dirt, oil, grease, and other contaminants.

There is demand from brand owners and consumers for development of alternative surfactants to those historically derived from petrochemicals. A proposed alternative has been sugar based surfactants. However, previously proposed sugar based surfactants have exhibited poor water solubility, especially in the presence of salts; and poor cleaning performance when used in single surfactant systems (e.g., without an anionic cosurfactant). Recent efforts have sought to overcome the poor cleaning performance by exploring sugar based surfactants with branched hydrophobes. However, these materials have lower water solubility than sugar based surfactants with linear hydrophobes, making delivery of the surfactants to substrates to be cleaned difficult.

Accordingly, there remains a need for water soluble compositions of sugar based surfactants with improved cleaning performance.

The present invention provides a mixture of alkyl polyglycosides, comprising: a linear alkyl polyglucoside of formula A

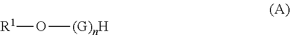

wherein R¹ is a linear C₆₋₁₅ alkyl group; wherein n is an average of 1 to 5; wherein G is a monosaccharide of glucose; and a branched alkyl polysaccharide of formula B

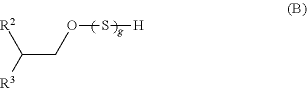

wherein R² is a C₂ alkyl group; wherein R³ is a linear or branched C₄ alkyl group; wherein g is an average of 1 to 5; wherein S is a monosaccharide with 5 carbon atoms;
with the proviso that R¹ is a linear C₉₋₁₅ alkyl group in at least some of the linear alkyl polyglucoside of formula A; and with the proviso that the mixture of alkyl polyglycosides comprises <3 wt %, based on weight of the mixture of alkyl polyglycosides, of a compound of formula IV

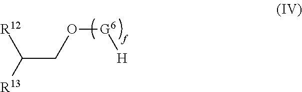

wherein R¹² is —(CH₂)ₚCH₃; wherein R¹³ is —(CH₂)ₚ₊₁CH(CH₃)₂; wherein G⁶ is a monosaccharide with 4 to 6 carbon atoms; wherein f is 1.1 to 10 and wherein p is 1 to 4.

The present invention provides a mixture of alkyl polyglycosides, comprising: a linear alkyl polyglucoside of formula A

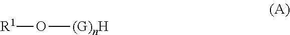

wherein R¹ is a linear C₆₋₁₅ alkyl group; wherein n is an average of 1 to 5; wherein G is a monosaccharide of glucose; and a branched alkyl polysaccharide of formula B

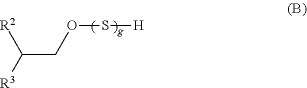

wherein R² is a C₂ alkyl group; wherein R³ is a linear or branched C₄ alkyl group; wherein g is an average of 1 to 5 and wherein S is a monosaccharide with 5 carbon atoms; with the proviso that R¹ is a linear C₉₋₁₅ alkyl group in at least some of the linear alkyl polyglucoside of formula A; and with the proviso that the mixture of alkyl polyglycosides comprises <3 wt %, based on weight of the mixture of alkyl polyglycosides, of a compound of formula IV

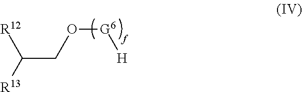

wherein R¹² is —(CH₂)ₚCH₃; wherein R¹³ is —(CH₂)ₚ₊₁CH(CH₃)₂; wherein G⁶ is a monosaccharide with 4 to 6 carbon atoms; wherein f is 1.1 to 10 and wherein p is 1 to 4.

The present invention provides an aqueous cleaning composition comprising water and a mixture of alkyl polyglycosides of the present invention.

The present invention provides a method of cleaning a hard surface, comprising: providing a soiled hard surface; wetting the soiled hard surface with an aqueous cleaning composition of the present invention; and wiping the wetted soiled hard surface to provide a cleaned hard surface.

### DETAILED DESCRIPTION

We have surprisingly found that a mixture of alkyl polyglycosides comprising a linear alkyl polyglucoside of formula A

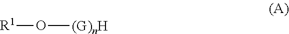

wherein R¹ is a linear C₆₋₁₅ alkyl group; wherein n is an average of 1 to 5; wherein G is a monosaccharide of glucose; and a branched alkyl polysaccharide of formula B

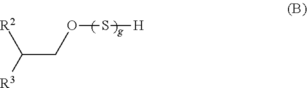

wherein R² is a C₂ alkyl group; wherein R³ is a linear or branched C₄ alkyl group; wherein g is an average of 1 to 5 and wherein S is a monosaccharide with 5 carbon atoms, as defined herein, is both water soluble and provides exceptional cleaning performance.

Unless otherwise indicated, ratios, percentages, parts, and the like are by weight (e.g., “ppm” means parts per million by weight).

The term “water soluble” as used herein and in the appended claims with respect to a given concentration of a mixture of alkyl polyglycosides in water, means that a solution of the given concentration of the mixture of alkyl polyglycosides in water will have a turbidity of <15 Nephelometric Turbidity Units (NTU) measured at 20° C. using a turbidity meter.

Preferably, the mixture of alkyl polyglycosides of the present invention comprises: a linear alkyl polyglucoside of formula A (preferably, 50 to 95 wt %; more preferably, 60 to 95 wt %; still more preferably, 65 to 92.5 wt %; most preferably, 67 to 90 wt %), based on weight of the mixture of alkyl polyglycosides, of the linear alkyl polyglucoside of formula A)

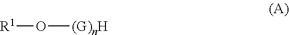

wherein R¹ is a linear C₆₋₁₅ alkyl group (preferably, a linear C₈₋₁₄ alkyl group; more preferably, a linear C₈₋₁₀ alkyl group; still more preferably, at least one of a linear C₈ alkyl group and a linear C₁₀ alkyl group; most preferably, a mixture of a linear C₈ alkyl group and a linear C₁₀ alkyl group); wherein n is an average of 1 to 5 (preferably, 1 to 3; more preferably, 1 to 2; most preferably, 1.1 to 2) and wherein G is a monosaccharide of glucose; and a branched alkyl polysaccharide of formula B (preferably, 5 to 50 wt %; more preferably, 5 to 40 wt %; still more preferably, 7.5 to 35 wt %; most preferably, 10 to 33 wt %), based on weight of the mixture of alkyl polyglycosides, of the branched alkyl polysaccharide of formula B)

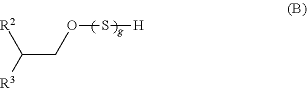

wherein R² is a C₂ alkyl group; wherein R³ is a linear or branched C₄ alkyl group (preferably, a linear C₄ alkyl group); wherein g is an average of 1 to 5 (preferably, 1 to 3; more preferably, 1 to 2; most preferably, 1.1 to 2) and wherein S is a monosaccharide with 5 carbon atoms (preferably, ribulose, xylulose, ribose, arabinose, xylose, lyxose and mixtures thereof; more preferably, arabinose, xylose and mixtures thereof; most preferably, xylose); with the proviso that R¹ is a linear C₉₋₁₅ alkyl group (preferably, a linear C₉₋₁₄ alkyl group; more preferably, a linear C₁₀₋₁₂ alkyl group; most preferably, a linear C₁₀ alkyl group) in at least some (preferably, ≥5 wt % (more preferably, ≥10 wt %; still more preferably, ≥25 wt %; yet more preferably, ≥40 wt %; most preferably, ≥45 wt %) of the linear alkyl polyglucoside of formula A; and with the proviso that the mixture of alkyl polyglycosides comprises <3 wt % (preferably, <0.5 wt %; more preferably, <0.1 wt %; still more preferably, <0.05 wt %; yet more preferably, <0.01 wt %; still yet more preferably, <0.001 wt %; yet still more preferably, <0.0001 wt %; most preferably, less than detectable limit), based on weight of the mixture of alkyl polyglycosides, of a compound of formula IV

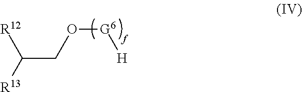

wherein R¹² is —(CH₂)ₚCH₃; wherein R¹³ is —(CH₂)ₚ₊₁CH(CH₃)₂; wherein G⁶ is a monosaccharide with 4 to 6 carbon atoms; wherein f is 1.1 to 10 and wherein p is 1 to 4.

Preferably, the mixture of alkyl polyglycosides of the present invention comprises: 50 to 95 wt % (preferably, 60 to 95 wt %; more preferably, 65 to 92.5 wt %; most preferably, 67 to 90 wt %), based on weight of the mixture of alkyl polyglycosides, of a linear alkyl polyglucoside of formula A; wherein R¹ is a linear C₆₋₁₅ alkyl group (preferably, a linear C₈₋₁₄ alkyl group; more preferably, a linear C₈₋₁₀ alkyl group; still more preferably, at least one of a linear C₈ alkyl group and a linear C₁₀ alkyl group; most preferably, a mixture of a linear C₈ alkyl group and a linear C₁₀ alkyl group); wherein n is an average of 1 to 5 (preferably, 1 to 3; more preferably, 1 to 2; most preferably, 1.1 to 2) and wherein G is a monosaccharide of glucose; and 5 to 50 wt % (preferably, 5 to 40 wt %; more preferably, 7.5 to 35 wt %; most preferably, 10 to 33 wt %), based on weight of the mixture of alkyl polyglycosides, of a branched alkyl polysaccharide of formula B; wherein R² is a C₂ alkyl group; wherein R³ is a linear or branched C₄ alkyl group (preferably, a linear C₄ alkyl group); wherein g is an average of 1 to 5 (preferably, 1 to 3; more preferably, 1 to 2; most preferably, 1.1 to 2) and wherein S is a monosaccharide with 5 carbon atoms (preferably, ribulose, xylulose, ribose, arabinose, xylose, lyxose and mixtures thereof; more preferably, arabinose, xylose and mixtures thereof; most preferably, xylose); with the proviso that R¹ is a linear C₉₋₁₅ alkyl group (preferably, a linear C₉₋₁₄ alkyl group; more preferably, a linear C₁₀₋₁₂ alkyl group; most preferably, a linear C₁₀ alkyl group) in at least some (preferably, ≥5 wt % (more preferably, ≥10 wt %; still more preferably, ≥25 wt %; yet more preferably, ≥40 wt %; most preferably, ≥45 wt %) of the linear alkyl polyglucoside of formula A; with the proviso that the mixture of alkyl polyglycosides comprises <3 wt % (preferably, <0.5 wt %; more preferably, <0.1 wt %; still more preferably, <0.05 wt %; yet more preferably, <0.01 wt %; still yet more preferably, <0.001 wt %; yet still more preferably, <0.0001 wt %; most preferably, less than detectable limit), based on weight of the mixture of alkyl polyglycosides, of a compound of formula IV

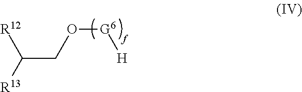

wherein R¹² is —(CH₂)ₚCH₃; wherein R¹³ is —(CH₂)ₚ₊₁CH(CH₃)₂; wherein G⁶ is a monosaccharide with 4 to 6 carbon atoms; wherein f is 1.1 to 10 and wherein p is 1 to 4; and with any one or more of the following additional provisos (i)-(vi): (i) with the proviso that the mixture of alkyl polyglycosides comprises <5 wt % (preferably, <1 wt %; more preferably, <0.1 wt %; still more preferably, <0.01 wt %; yet more preferably, 0.001 wt %; still yet more preferably, 0.0001 wt %; most preferably, less than detectable limit), based on weight of the mixture of alkyl polyglycosides, of a compound of formula I

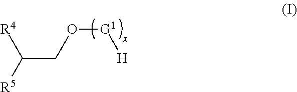

wherein R⁴ is a linear or branched C₃₋₄ alkyl group; wherein R⁵ is a linear or branched C₅₋₆ alkyl group; wherein G¹ is a monosaccharide with 4 to 6 carbon atoms and wherein x is 1.1 to 4; (ii) with the proviso that the mixture of alkyl polyglycosides comprises <1 wt % (preferably, <0.5 wt %; more preferably, <0.1 wt %; still more preferably, <0.01 wt %; yet more preferably, <0.001 wt %; still yet more preferably, <0.0001 wt %; most preferably, less than detectable limit), based on weight of the mixture of alkyl polyglycosides, of an alkyl oligoglucoside of formula II

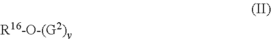

wherein R¹⁶ is a branched C₉₋₁₁ alkyl group; wherein G² is a glucose unit and wherein y is 1.4 to 2.0; (iii) with the proviso that the mixture of alkyl polyglycosides comprises <10 wt % (preferably, <5 wt %; more preferably, <1 wt %; still more preferably, <0.1 wt %; yet more preferably, <0.01 wt %; still yet more preferably, <0.001 wt %; yet still more preferably, <0.0001 wt %; most preferably, less than detectable limit), based on weight of the mixture of alkyl polyglycosides, of a compound of formula III

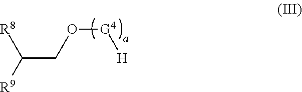

wherein R⁸ is —(CH₂)_{b}CH(CH₃)₂; wherein R⁹ is —(CH₂)_{b+2}CH(CH₃)₂; G⁴ is a monosaccharide with 4 to 6 carbon atoms; wherein a is 1.1 to 4 and wherein b is 0 to 3; (iv) with the proviso that the linear alkyl polyglucoside of formula A and the branched alkyl polysaccharide of formula B comprise 95 to 100 wt % (preferably, 97 to 100 wt %; more preferably, 98 to 100 wt %; still more preferably, 99 to 100 wt %; yet more preferably, 99.5 to 100 wt %; still yet more preferably, 99.9 to 100 wt %; most preferably, 100 wt %) of the alkyl polyglycosides present in the mixture of alkyl polyglycosides; (v) with the proviso that the mixture of alkyl polyglycosides comprises <0.05 wt % (preferably, <0.01; more preferably, <0.005; still more preferably, <0.001; yet more preferably, <0.0005; still yet more preferably, <0.0001 wt %; most preferably, less than detectable limit), based on weight of the mixture of alkyl polyglycosides, of a fatty acid alkyl ester alkoxylate of formula V

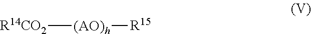

wherein R¹⁴ is a linear or branched, saturated or unsaturated, alkyl group containing 5 to 21 carbon atoms; wherein AO is a C₂₋₄ alkylene oxide unit, wherein h is 1 to 30 and wherein R¹⁵ is a linear or branched alkyl group containing 1 to 6 carbon atoms; and/or (vi) with the proviso that when the mixture of alkyl polyglycosides comprises both fatty acid ester C₁₂₋₂₃ alkyl-(O)O-moieties and fatty acid ester C₆₋₁₀ alkyl-(O)O-moieties, the mole ratio of the C₁₂₋₂₃ alkyl-(O)O-moieties to the C₆₋₁₀ alkyl-(O)O-moieties is >1.1:1 (preferably, >1.2:1; more preferably, >1.3:1; still more preferably, >1.5:1; most preferably, >2:1).

Preferably, the mixture of alkyl polyglycosides of the present invention comprises 50 to 95 wt % (preferably, 60 to 95 wt %; more preferably, 65 to 92.5 wt %; most preferably, 67 to 90 wt %), based on weight of the mixture of alkyl polyglycosides, of a linear alkyl polyglucoside of formula A; wherein R¹ is a linear C₆₋₁₅ alkyl group (preferably, a linear C₈₋₁₄ alkyl group; more preferably, a linear C₈₋₁₀ alkyl group; still more preferably, at least one of a linear C₈ alkyl group and a linear C₁₀ alkyl group; most preferably, a mixture of a linear C₈ alkyl group and a linear C₁₀ alkyl group); wherein n is an average of 1 to 5 (preferably, 1 to 3; more preferably, 1 to 2; most preferably, 1.1 to 2); wherein G is a monosaccharide of glucose. More preferably, the mixture of alkyl polyglycosides of the present invention comprises 50 to 95 wt % (preferably, 60 to 95 wt %; more preferably, 65 to 92.5 wt %; most preferably, 67 to 90 wt %), based on weight of the mixture of alkyl polyglycosides, of a linear alkyl polyglucoside of formula A; wherein the linear alkyl polyglucoside of formula A is a blend comprising a linear C₈ alkyl polyglucoside and a linear C₁₀ alkyl polyglucoside. Most preferably, the mixture of alkyl polyglycosides of the present invention comprises 50 to 95 wt % (preferably, 60 to 95 wt %; more preferably, 65 to 92.5 wt %; most preferably, 67 to 90 wt %), based on weight of the mixture of alkyl polyglycosides, of a linear alkyl polyglucoside of formula A; wherein the linear alkyl polyglucoside of formula A is a blend comprising 5 to 95 wt % (preferably, 10 to 90 wt %; more preferably, 25 to 75 wt %; still more preferably, 40 to 60 wt %; most preferably, 45 to 55 wt %), based on weight of the blend, of a linear C₈ alkyl polyglucoside and 5 to 95 wt % (preferably, 10 to 90 wt %; more preferably, 25 to 75 wt %; still more preferably, 40 to 60 wt %; most preferably, 45 to 55 wt %), based on weight of the blend, of a linear C₁₀ alkyl polyglucoside.

Preferably, the mixture of alkyl polyglycosides of the present invention comprises 5 to 50 wt % (preferably, 5 to 40 wt %; more preferably, 7.5 to 35 wt %; most preferably, 10 to 33 wt %), based on weight of the mixture of alkyl polyglycosides, of a branched alkyl polysaccharide of formula B; wherein R² is a C₂ alkyl group; wherein R³ is a linear or branched C₄ alkyl group (preferably, a linear C₄ alkyl group); wherein g is an average of 1 to 5 (preferably, 1 to 3; more preferably, 1 to 2; most preferably, 1.1 to 2); wherein S is a monosaccharide with 5 carbon atoms (preferably, ribulose, xylulose, ribose, arabinose, xylose, lyxose and mixtures thereof; more preferably, arabinose, xylose and mixtures thereof; most preferably, xylose). More preferably, the mixture of alkyl polyglycosides of the present invention comprises 5 to 50 wt % (preferably, 5 to 40 wt %; more preferably, 7.5 to 35 wt %; most preferably, 10 to 33 wt %), based on weight of the mixture of alkyl polyglycosides, of a branched alkyl polysaccharide of formula B; wherein R² is a C₂ alkyl group; wherein R³ is a linear or branched C₄ alkyl group (preferably, a linear C₄ alkyl group); wherein g is an average of 1 to 5 (preferably, 1 to 3; more preferably, 1 to 2; most preferably, 1.1 to 2); wherein S is a monosaccharide with 5 carbon atoms (preferably, ribulose, xylulose, ribose, arabinose, xylose, lyxose and mixtures thereof; more preferably, arabinose, xylose and mixtures thereof; most preferably, xylose). Most preferably, the mixture of alkyl polyglycosides of the present invention comprises 5 to 50 wt % (preferably, 5 to 40 wt %; more preferably, 7.5 to 35 wt %; most preferably, 10 to 33 wt %), based on weight of the mixture of alkyl polyglycosides, of a branched alkyl polysaccharide of formula B; wherein R² is a C₂ alkyl group; wherein R³ is a linear or branched C₄ alkyl group (preferably, a linear C₄ alkyl group); wherein g is an average of 1 to 5 (preferably, 1 to 3; more preferably, 1 to 2; most preferably, 1.1 to 2); wherein S is a monosaccharide with 5 carbon atoms (preferably, ribulose, xylulose, ribose, arabinose, xylose, lyxose and mixtures thereof; more preferably, arabinose, xylose and mixtures thereof; most preferably, xylose); and wherein the branched alkyl polysaccharide of formula B comprises >90 wt % (preferably, 92 to 100 wt %; more preferably, 95 to 100 wt %; still more preferably, 98 to 100 wt %; most preferably, 99 to 100 wt %), based on weight of the branched alkyl polysaccharide of formula B, of branched alkyl polysaccharide of formula B where S is xylose.

Preferably, the aqueous cleaning composition of the present invention, comprises: water (preferably, 25 to 99.9 wt % (more preferably, 50 to 99.5 wt %; still more preferably, 60 to 99.25 wt %; most preferably, 65 to 99 wt %), based on weight of the aqueous cleaning composition, of the water); and a mixture of alkyl polyglycosides of the present invention (preferably, 0.1 to 15 wt % (more preferably, 0.5 to 10 wt %; still more preferably, 0.75 to 7.5 wt %; most preferably, 1 to 7 wt %), based on weight of the aqueous cleaning composition, of the mixture of alkyl polyglycosides) (preferably, wherein the mixture of alkyl polyglycosides is water soluble). More preferably, the aqueous cleaning composition of the present invention, comprises: water (preferably, 25 to 99.9 wt % (more preferably, 50 to 99.5 wt %; still more preferably, 60 to 99.25 wt %; most preferably, 65 to 99 wt %), based on weight of the aqueous cleaning composition, of the water); and a mixture of alkyl polyglycosides of the present invention (preferably, 0.1 to 15 wt % (more preferably, 0.5 to 10 wt %; still more preferably, 0.75 to 7.5 wt %; most preferably, 1 to 7 wt %), based on weight of the aqueous cleaning composition, of the mixture of alkyl polyglycosides) (preferably, wherein the mixture of alkyl polyglycosides is water soluble); with any one or more of the following provisos (i)-(v): (i) with the proviso that the mixture of alkyl polyglycosides comprises 90 to 100 wt % (preferably, 95 to 100 wt %; more preferably, 97.5 to 100 wt %; still more preferably, 98 to 100 wt %; yet more preferably, 99 to 100 wt %; still yet more preferably, 99.5 to 100 wt %; yet still more preferably, 99.9 to 100 wt %; most preferably, 100 wt %), based on weight of the aqueous cleaning composition, of all alkyl polyglucosides in the aqueous cleaning composition; (ii) with the proviso that the aqueous cleaning composition comprises <0.05 wt % (preferably, <0.01 wt %; more preferably, <0.005 wt %; still more preferably, <0.001 wt %; yet more preferably, <0.0005 wt %; still yet more preferably, <0.0001 wt %; yet still more preferably, <0.00001 wt %; most preferably, less than detectable limit), based on weight of the aqueous cleaning composition, of a compound of formula III

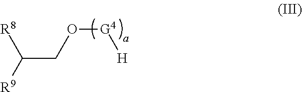

wherein R⁸ is —(CH₂)_{b}CH(CH₃)₂; wherein R⁹ is —(CH₂)_{b+2}CH(CH₃)₂; G⁴ is a monosaccharide with 4 to 6 carbon atoms; wherein a is 1.1 to 4 and wherein b is 0 to 3; (iii) with the proviso that the aqueous cleaning composition comprises <0.05 wt % (preferably, <0.01; more preferably, <0.005; still more preferably, <0.001; yet more preferably, <0.0005; still yet more preferably, <0.0001 wt %; most preferably, less than detectable limit), based on weight of the aqueous cleaning composition, of a fatty acid alkyl ester alkoxylate of formula V

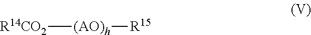

wherein R¹⁴ is a linear or branched, saturated or unsaturated, alkyl group containing 5 to 21 carbon atoms; wherein AO is a C₂₋₄ alkylene oxide unit, wherein h is 1 to 30 and wherein R¹⁵ is a linear or branched alkyl group containing 1 to 6 carbon atoms; (iv) with the proviso that the aqueous cleaning composition comprises <0.01 wt % (preferably, <0.005 wt %; more preferably, <0.001 wt %; still more preferably, <0.0001 wt %; yet more preferably, <0.00001 wt %; most preferably, less than detectable limit), based on weight of the aqueous cleaning composition, of alkyl polyglycoside comprising fatty acid ester C₁₂₋₂₃ alkyl-(O)O-moieties and/or fatty acid ester C₆₋₁₀ alkyl-(O)O-moieties; and/or (v) with the proviso that when the aqueous cleaning composition comprises both an anionic surfactant and a nonionic surfactant, the weight ratio of the anionic surfactant to the nonionic surfactant in the aqueous cleaning composition is greater than 1:1 (preferably, >2:1; more preferably >2.5:1); wherein the mixture of alkyl polyglycosides is not considered a nonionic surfactant for purposes of the weight ratio of anionic surfactant to nonionic surfactant (i.e., the anionic cleaning surfactant predominates over any nonionic surfactant present, excluding the mixture of alkyl polyglycosides) (i.e., the mixture of alkyl polyglycosides is not considered a nonionic surfactant for purposes of calculating the weight ratio of anionic to nonionic surfactant).

Preferably, the aqueous cleaning composition of the present invention, comprises 25 to 99.9 wt % (preferably, 50 to 99.5 wt %; more preferably, 60 to 99.25 wt %; most preferably, 65 to 99 wt %), based on weight of the aqueous cleaning composition, of water. More preferably, the aqueous cleaning composition of the present invention, comprises 25 to 99.9 wt % (preferably, 50 to 99.5 wt %; more preferably, 60 to 99.25 wt %; most preferably, 65 to 99 wt %), based on weight of the aqueous cleaning composition, of water; wherein the water comprises at least one of distilled water, deionized water and industrial soft water.

Preferably, the aqueous cleaning composition of the present invention, further comprises an additive. Preferably, the aqueous cleaning composition of the present invention, further comprises an additive; wherein the additive is selected from the group consisting of a cleaning surfactant (other than the mixture of alkyl polyglycosides of the present invention), a dispersant polymer, a salt (e.g., sodium chloride, magnesium sulfate), a builder, an enzyme, a corrosion inhibitor, an acid, a base (e.g., sodium hydroxide), a bleaching agent, an abrasive, an antimicrobial agent (e.g., phenoxyethanol), a chelating agent, a pH adjuster, a buffering agent, a dye, a fragrance, a hydrotrope, an organic solvent, a rheology modifier, sulfonated polymer, poly(alkylene oxide) polymer and mixtures thereof.

Preferably, the aqueous cleaning composition of the present invention optionally further comprises a cleaning surfactant other than the mixture of alkyl polyglycosides of the present invention; wherein the cleaning surfactant is selected from the group consisting of nonionic surfactants other than the mixture of alkyl polyglycosides, anionic surfactants, cationic surfactants, zwitterionic surfactants, amphoteric surfactants and mixtures thereof. Preferably, the aqueous cleaning composition of the present invention comprises 0.01 to 35 wt % (preferably, 0.1 to 20 wt %; more preferably, 1 to 15 wt %; most preferably, 5 to 12 wt %), based on the weight of the aqueous cleaning composition, of a cleaning surfactant other than the mixture of alkyl polyglycosides of the present invention; wherein the cleaning surfactant is selected from the group consisting of nonionic surfactants other than the mixture of alkyl polyglycosides, anionic surfactants, cationic surfactants, zwitterionic surfactants, amphoteric surfactants and mixtures thereof. Still more preferably, the aqueous cleaning composition of the present invention comprises 0.01 to 35 wt % (preferably, 0.1 to 20 wt %; more preferably, 1 to 15 wt %; most preferably, 5 to 12 wt %), based on the weight of the aqueous cleaning composition, of a cleaning surfactant other than the mixture of alkyl polyglycosides of the present invention; wherein the cleaning surfactant includes an anionic surfactant. Most preferably, the aqueous cleaning composition of the present invention comprises 0.01 to 35 wt % (preferably, 0.1 to 20 wt %; more preferably, 1 to 15 wt %; most preferably, 5 to 12 wt %), based on the weight of the aqueous cleaning composition, of a cleaning surfactant other than the mixture of alkyl polyglycosides of the present invention; wherein the cleaning surfactant includes a mixture of anionic surfactants.

Preferably, the anionic surfactant is selected from the group consisting of alkyl sulfonic acids, alkyl sulfates, alkyl sulfonates, alkyl benzene sulfonic acid, alkyl benzene sulfates, alkyl benzene sulfonates, alkyl ether sulfonic acids, alkyl ether sulfates, alkyl ether sulfonates, paraffin sulfonic acids, paraffin sulfates, paraffin sulfonates, olefin sulfonic acids, olefin sulfates, olefin sulfonates, alpha-sulfocarboxylates, esters of alpha-sulfocarboxylates, alkyl glyceryl ether sulfonic acids, alkyl glyceryl ether sulfates, alkyl glyceryl ether sulfonates, sulfates of fatty acids, sulfonates of fatty acids, sulfonates of fatty acid esters, alkyl phenol polyethoxy ether sulfates, 2-acryloxy-alkane-1-sulfonic acid, 2-acryloxy-alkane-1-sulfonate, beta-alkyloxy alkane sulfonic acid, beta-alkyloxy alkane sulfonate, salts thereof and mixtures thereof. More preferably, the anionic surfactant is selected from the group consisting of C₈₋₂₀ alkyl benzene sulfonic acid, C₈₋₂₀ alkyl benzene sulfates, C₈₋₂₀ alkyl benzene sulfonate, C₈₋₂₀ alkyl ether sulfonic acids, C₈₋₂₀ alkyl ether sulfates, C₈₋₂₀ alkyl ether sulfonates, paraffin sulfonic acid, paraffin sulfates, paraffin sulfonate, alpha-olefin sulfonic acid, alpha-olefin sulfate, alpha-olefin sulfonate, sulfonates of fatty acids, sulfonates of fatty acid esters, salts thereof and mixtures thereof. Still more preferably the anionic surfactant is selected from the group consisting of C₁₁₋₁₃ alkyl benzene sulfonic acid, C₁₁₋₁₃ alkyl benzene sulfonate, C₁₁₋₁₃ alkyl polyethoxy sulfonic acids, C₁₁₋₁₃ alkyl polyethoxy sulfates, C₁₁₋₁₃ alkyl polyethoxy sulfonates, salts thereof and mixtures thereof. Yet more preferably the anionic surfactants is selected from the group consisting of C₁₀₋₁₆ alkyl benzene sulfonic acid, C₁₀₋₁₆ alkyl benzene sulfonate, C₁₀₋₁₆ alkyl polyethoxy sulfonic acids, C₁₀₋₁₆ alkyl polyethoxy sulfates, C₁₀₋₁₆ alkyl polyethoxy sulfonates, salts thereof and mixtures thereof. Most preferably the anionic surfactant includes (preferably, is) a mixture of sodium lauryl ether sulfate (SLES) and C₁₀₋₁₆ alkyl benzene sulfonic acid or salt thereof.

Preferably, the amphoteric surfactant is selected from the group consisting of betaines, amine oxides, alkylamidoalkylamines, alkyl-substituted amine oxides, acylated amino acids, derivatives of aliphatic quaternary ammonium compounds and mixtures thereof. More preferably, the amphoteric surfactant includes a betaine. Most preferably, the amphoteric surfactant is a cocamidopropyl betaine.

Non-ionic surfactants other than the mixture of alkyl polyglycosides of the invention, include alkoxylates, polyglycol ethers, fatty alcohol polyglycol ethers, alkylphenol polyglycol ethers, end group capped polyglycol ethers, mixed ethers, hydroxy mixed ethers, fatty acid polyglycol esters and mixtures thereof.

Preferably, the aqueous cleaning composition of the present invention optionally further comprises an organic solvent. More preferably, the aqueous cleaning composition of the present invention optionally further comprises 0 to 10 wt % (preferably, 0.1 to 10 wt %; more preferably, 0.5 to 7.5 wt %; most preferably, 1 to 5 wt %), based on the weight of the aqueous cleaning composition, of an organic solvent. Preferably, the aqueous cleaning composition of the present invention optionally further comprises 0 to 10 wt % (preferably, 0.1 to 10 wt %; more preferably, 0.5 to 7.5 wt %; most preferably, 1 to 5 wt %), based on the weight of the aqueous cleaning composition, of an organic solvent; wherein the organic solvent is miscible with water. More preferably, the aqueous cleaning composition of the present invention optionally further comprises 0 to 10 wt % (preferably, 0.1 to 10 wt %; more preferably, 0.5 to 7.5 wt %; most preferably, 1 to 5 wt %), based on the weight of the aqueous cleaning composition, of an organic solvent; wherein the organic solvent is selected from the group consisting of an aliphatic alcohol (e.g., C₁₋₆ alkanols, C₁₋₆ alkyl diols); a monoalkylene glycol ether (e.g., ethylene glycol propyl ether, ethylene glycol n-butyl ether, ethylene glycol t-butyl ether, propylene glycol propyl ether, propylene glycol n-butyl ether, propylene glycol t-butyl ether, propylene glycol methyl ether acetate, propylene glycol diacetate); a polyalkylene glycol ether (e.g., diethylene glycol ethyl ether, diethylene glycol propyl ether, diethylene glycol n-butyl ether, diethylene glycol t-butyl ether, diethylene glycol hexyl ether, dipropylene glycol methyl ether, dipropylene glycol ethyl ether, dipropylene glycol propyl ether, dipropylene glycol n-butyl ether, dipropylene glycol t-butyl ether, dipropylene glycol phenyl ether, dipropylene glycol methyl ether acetate, tripropylene glycol methyl ether, tripropylene glycol ethyl ether, tripropylene glycol propyl ether, tripropylene glycol n-butyl ether, tripropylene glycol t-butyl ether) and mixtures thereof. Still more preferably, the aqueous cleaning composition of the present invention optionally further comprises 0 to 10 wt % (preferably, 0.1 to 10 wt %; more preferably, 0.5 to 7.5 wt %; most preferably, 1 to 5 wt %), based on the weight of the aqueous cleaning composition, of an organic solvent; wherein the organic solvent is selected from the group consisting of isopropanol, ethanol, 2-(2-butoxyethoxy) ethanol, ethylene glycol butyl ether, propylene glycol methyl ether, propylene glycol propyl ether, propylene glycol t-butyl ether, dipropylene glycol methyl ether, dipropylene glycol propyl ether, dipropylene glycol n-butyl ether and mixtures thereof. Yet more preferably, the aqueous cleaning composition of the present invention optionally further comprises 0 to 10 wt % (preferably, 0.1 to 10 wt %; more preferably, 0.5 to 7.5 wt %; most preferably, 1 to 5 wt %), based on the weight of the aqueous cleaning composition, of an organic solvent; wherein the organic solvent includes ethanol. Most preferably, the aqueous cleaning composition of the present invention optionally further comprises 0 to 10 wt % (preferably, 0.1 to 10 wt %; more preferably, 0.5 to 7.5 wt %; most preferably, 1 to 5 wt %), based on the weight of the aqueous cleaning composition, of an organic solvent; wherein the organic solvent is ethanol.

Preferably, the aqueous cleaning composition of the present invention further comprises a dispersant polymer. More preferably, the aqueous cleaning composition of the present invention further comprises 0.01 to 2 wt % (preferably, 0.05 to 1.5 wt %; more preferably, 0.075 to 1 wt %; most preferably, 0.1 to 0.75 wt %), based on weight of the aqueous cleaning composition, of a dispersant polymer. Most preferably, the aqueous cleaning composition of the present invention further comprises 0.01 to 2 wt % (preferably, 0.05 to 1.5 wt %; more preferably, 0.075 to 1 wt %; most preferably, 0.1 to 0.75 wt %), based on weight of the aqueous cleaning composition, of a dispersant polymer; wherein the dispersant polymer is a polyacrylic acid homopolymer. Preferably, the polyacrylic acid homopolymer has a molecular weight of 1,000 to 7,500 Daltons (preferably, ≥1,500 Daltons; more preferably, ≥2,000 Daltons; still more preferably, ≥3,000 Daltons; most preferably, ≥4,000 Daltons; preferably, ≤7,500 Daltons; more preferably, ≤7,000 Daltons; still more preferably, ≤6,000 Daltons; most preferably, ≤5,000 Daltons).

Preferably, the aqueous cleaning composition of the present invention, further comprises: 0 to 10 wt % (preferably, 0.1 to 10 wt %; more preferably, 0.5 to 7.5 wt %; most preferably, 1 to 5 wt %), based on weight of the aqueous cleaning composition, of a hydrotrope. More preferably, the aqueous cleaning composition of the present invention, optionally further comprises: 0 to 10 wt % (preferably, 0.1 to 10 wt %; more preferably, 0.5 to 7.5 wt %; most preferably, 1 to 5 wt %), based on weight of the aqueous cleaning composition, of a hydrotrope; wherein the hydrotrope is selected from the group consisting of urea; tosylate; calcium, sodium, potassium, ammonium and alkanol ammonium salts of (i) xylene sulfonic acid, (ii) toluene sulfonic acid, (iii) ethylbenzene sulfonic acid and (iv) cumene sulfonic acid; and mixtures thereof. Still more preferably, the aqueous cleaning composition of the present invention, further comprises: 0 to 10 wt % (preferably, 0.1 to 10 wt %; more preferably, 0.5 to 7.5 wt %; most preferably, 1 to 5 wt %), based on weight of the aqueous cleaning composition, of a hydrotrope; wherein the hydrotrope is selected from the group consisting of sodium toluene sulfonate, potassium toluene sulfonate, sodium xylene sulfonate, ammonium xylene sulfonate, potassium xylene sulfonate, calcium xylene sulfonate, sodium cumene sulfonate, ammonium cumene sulfonate and mixtures thereof. Yet more preferably, the aqueous cleaning composition of the present invention, further comprises: 0 to 10 wt % (preferably, 0.1 to 10 wt %; more preferably, 0.5 to 7.5 wt %; most preferably, 1 to 5 wt %), based on weight of the aqueous cleaning composition, of a hydrotrope; wherein the hydrotrope includes sodium xylene sulfonate. Most preferably, the aqueous cleaning composition of the present invention, further comprises: 0 to 10 wt % (preferably, 0.1 to 10 wt %; more preferably, 0.5 to 7.5 wt %; most preferably, 1 to 5 wt %), based on weight of the aqueous cleaning composition, of a hydrotrope; wherein the hydrotrope is sodium xylene sulfonate.

Preferably, the aqueous cleaning composition of the present invention is selected from the group consisting of a hard surface cleaning formulation and a hand dishwashing liquid. More preferably, the aqueous cleaning composition of the present invention is a hard surface cleaning formulation.

Preferably, the method of cleaning a hard surface of the present invention, comprises: providing a soiled hard surface (preferably, wherein the soiled hard surface is selected from the group consisting of a counter top surface, a cook top surface, a ceramic surface, a laminate surface, a porcelain surface, a tile surface, a glass surface, a metal surface, a wood surface (preferably, wherein the soiled hard surface is a nonporous surface; more preferably, wherein the soiled hard surface is a soiled tile)) (preferably, the soiled hard surface is soiled with at least one of an oily soil or a greasy soil); wettin the soiled hard surface with an aqueous cleaning composition of the present invention; and wiping the wetted soiled hard surface (preferably with an implement selected from the group consisting of a cloth, towel, wipe, brush, mop, wiper, squeegee, sponge or a combination thereof) to provide a cleaned hard surface.

Some embodiments of the present invention will now be described in detail in the following Examples.

### Syntheses S1-S3: Alkyl Polyglycosides

A round-bottomed reactor was placed in an aluminum heating block and fitted with a 25 mL Dean-Stark trap (containing 15 mL hexanes) and a magnetic stir bar. To the reactor was added Sugar and n-butanol as noted in TABLE 1. The reactor contents were then stirred vigorously under a nitrogen blanket (bubbler). To the stirring reactor contents was added para-toluenesulfonic acid monohydrate (1 mol %) and the aluminum heating block set at temperature T¹ (noted in TABLE 1) and maintained at that temperature until the rate of increase in water volume slowed to zero, for ~2-3 hours. The reactor contents were then allowed to cool to ambient temperature with continued stirring. Then Alcohol B (B molar equivalents) was added to the reactor contents as noted in TABLE 1. The Dean-Stark trap was then replated with a still head and a condenser flask. The reactor contents were then vacuum distilled with the aluminum heating block set at temperature T² (noted in TABLE 1) and the condenser flask set in an ice-cooled bath. The vacuum was periodically adjusted to a flow rate of 1-2 drops/second. The vacuum was maintained for 2 to 4 hours. The distillation was then stopped. The distillation apparatus was placed back under a nitrogen atmosphere and the stirred contents of the reactor were allowed to cool to ambient temperature. A solution of sodium hydroxide was added to the stirring reactor contents as noted in TABLE 1. After vigorous stirring, the pH was measured (pH paper) to ensure the reactor contents were basic. A sample of the reactor contents was diluted with hexanes and purified via automated column chromatography. The alcohol eluted at a mobile phase concentration of 100% hexanes to 20/80 ethyl acetate/hexanes. The solvent mixture was then ramped over 2 column volumes to 100% ethyl acetate. The solvent system was then changed to ethyl acetate-methanol, and the solvent ratio was ramped over 1-6 column volumes to 60 to 100% methanol—during which time the product eluted as a mixture of mono- and polysaccharides. The solvent was removed under reduced pressure to give the final product.

TABLE 1

| | Sugar | | n-BuOH | Alcohol B | | Temp ° C. | |
|---|---|---|---|---|---|---|---|
| Ex | Type | (g) | (eq) | Type | (eq) | T¹ | T² |
| S1 | xylose | 30 | 6 | 2-ethyl-1-hexan-1-ol | 3 | 137 | 135 |
| S2 | glucose | 10 | 5 | 2-butyloctan-1-ol | 4 | 120 | 130 |
| S3 | glucose | 18 | 5 | 2-ethyl-1-hexan-1-ol | 4 | 130 | 130 |

### Comparative Examples CF1-CF17 and Examples F1-F8: Cleaning Composition

An aqueous cleaning composition was prepared in each of Comparative Examples CF1-CF4 and Examples F1-F3 having a 5 wt % concentration in water of the alkyl polyglycoside or alkyl polyglycoside mixture as noted in TABLE 2.

An aqueous cleaning composition was prepared in each of Comparative Examples CF5-CF17 and Examples F4-F8 having a 1 wt % concentration in water of the alkyl polyglycoside mixture as noted in TABLE 3.

### Turbidity

The turbidity of the aqueous cleaning compositions prepared according to Comparative Examples CF1-CF17 and Examples F1-F8 was measured using a turbidity meter at 20° C. The meter was calibrated according to instructions included with the machine. The results of the turbidity measurements are provided in TABLES 2-3. The aqueous cleaning compositions having a measured turbidity of <15 Nephelometric Turbidity Units (NTU) are marked as clear; and those with a turbidity of >15 NTU are marked as turbid.

TABLE 2

| Alkyl polyglycoside mixture | | |
|---|---|---|
| Branched | Linear | Turbidity |

| Example | Type | (wt %) | Type | (wt %) | (NTU) |
|---|---|---|---|---|---|
| CF1 | — | — | A | 100 | Clear |
| CF2 | S3 | 10 | A | 90 | Clear |
| CF3 | S3 | 20 | A | 80 | Clear |
| CF4 | S3 | 30 | A | 70 | Clear |
| F1 | S1 | 10 | A | 90 | Clear |
| F2 | S1 | 20 | A | 80 | Clear |
| F3 | S1 | 30 | A | 70 | Clear |

^{A} TRITON ™ CG-50 linear C₈₋₁₀ alkyl polyglucoside from The Dow Chemical Company

TABLE 3

| Alkyl polyglycoside mixture | | |
|---|---|---|
| Branched | Linear | Turbidity |

| Example | Type | (wt %) | Type | (wt %) | (NTU) |
|---|---|---|---|---|---|
| CF5 | S1 | 60 | A | 40 | Turbid |
| CF6 | S1 | 70 | A | 30 | Turbid |
| CF7 | S1 | 80 | A | 20 | Turbid |
| CF8 | S1 | 90 | A | 10 | Turbid |
| CF9 | S2 | 10 | A | 90 | Clear |
| CF10 | S2 | 20 | A | 80 | Turbid |
| CF11 | S2 | 30 | A | 70 | Turbid |
| CF12 | S2 | 40 | A | 60 | Turbid |
| CF13 | S2 | 50 | A | 50 | Turbid |
| CF14 | S2 | 60 | A | 40 | Turbid |
| CF15 | S2 | 70 | A | 30 | Turbid |
| CF16 | S2 | 80 | A | 20 | Turbid |
| CF17 | S2 | 90 | A | 10 | Turbid |
| F4 | S1 | 10 | A | 90 | Clear |
| F5 | S1 | 20 | A | 80 | Clear |
| F6 | S1 | 30 | A | 70 | Clear |
| F7 | S1 | 40 | A | 60 | Clear |
| F8 | S1 | 50 | A | 50 | Clear |

^{A} TRITON ™ CG-50 linear C₈₋₁₀ alkyl polyglucoside from The Dow Chemical Company

### Cleaning Performance

The cleaning performance of the aqueous hard surface cleaning formulations prepared according to Comparative Examples CF1-CF4 and Examples F1-F3 was determined using the following procedure. A tile (Premium Excelon Feature Tile & Strip, Chalk II 56830/L597A available from ArmstrongFlooring) was soiled with 500 μL of a naptha and carbon black based dirt. The dirt was prepared by mixing 61.04 wt % Naptha 60/110, 27.6 wt % Vognis Myritol 318, 8.2 wt % soybean oil and 3.18 wt % Mogyul L carbon black using a stirbar in a beaker until all the carbon black was suspended. The dirt was then applied to the center of the tile and painted using a sponge applicator through 20 up and down movements while holding the tile at a 45 to 90° angle relative to the ground. The tile was then left to dry in overnight. The tile was then placed in a device for retaining a nonporous substrate as described in U.S. Patent Application Publication No. 20100083778, the disclosure of which is incorporated herein by reference in its entirety, wherein the removable well insert had 24 openings. The individual openings were filled with 400 μL of aqueous hard surface cleaning formulation and capped with 3 mm needle punch carpet purchased from The Felt Store Inc. (West Seneca, NY) adhered to 12.7 g conic weight. The plate and tile were mechanically moved laterally back and forth relative to one another to scrub the surface of the tile for 5 minutes. The plate was then removed and the surface of the tile was rinsed with deionized water. The surface of the scrubbed tile was then analyzed using an EPSON Perfection V500 Photo Scanner and ImageJ software to extract the gray value of each well on the scrubbed tile for each formulation. The gray value for each scrubbed tile region was then compared with the gray values measured for the original tile before soiling and the soiled region of the tile before cleaning. Note that a value of 0% corresponds with a gray value equal to that of the soiled region before cleaning and a value of 100% would correspond with a gray value equal to that of the original tile before soiling. The results are provided in TABLE 4.

TABLE 4

| Alkyl polyglycoside mixture | | |
|---|---|---|
| Branched | Linear | % soil |

| Example | Type | (wt %) | Type | (wt %) | removal |
|---|---|---|---|---|---|
| CF1 | — | — | A | 100 | 20 ± 2 |
| CF2 | S3 | 10 | A | 90 | 28 ± 5 |
| CF3 | S3 | 20 | A | 80 | 21 ± 8 |
| CF4 | S3 | 30 | A | 70 | 24 ± 2 |
| F1 | S1 | 10 | A | 90 | 51 ± 1 |
| F2 | S1 | 20 | A | 80 | 59 ± 2 |
| F3 | S1 | 30 | A | 70 | 64 ± 2 |

^{A} TRITON ™ CG-50 linear C₈₋₁₀ alkyl polyglucoside from The Dow Chemical Company

We claim:

1. A mixture of alkyl polyglycosides, comprising:
a linear alkyl polyglucoside of formula A 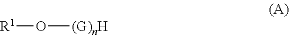
wherein R¹ is a linear C₆₋₁₅ alkyl group; wherein n is an average of 1 to 5; wherein G is a monosaccharide of glucose; and
a branched alkyl polysaccharide of formula B 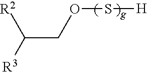
wherein R² is a C₂ alkyl group; wherein R³ is a linear or branched C₄ alkyl group; wherein g is an average of 1 to 5 and wherein S is a monosaccharide with 5 carbon atoms;
with the proviso that R¹ is a linear C₉₋₁₅ alkyl group in at least some of the linear alkyl polyglucoside of formula A; and
with the proviso that the mixture of alkyl polyglycosides comprises <3 wt %, based on weight of the mixture of alkyl polyglycosides, of a compound of formula IV 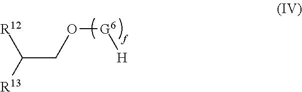
wherein R¹² is —(CH₂)ₚCH₃; wherein R¹³ is —(CH₂)ₚ₊₁CH(CH₃)₂; wherein G⁶ is a monosaccharide with 4 to 6 carbon atoms; wherein f is 1.1 to 10 and wherein p is 1 to 4.

2. The mixture of alkyl polyglycosides of claim 1, comprising 5 to 50 wt %, based on weight of the mixture of alkyl polyglycosides, of the branched alkyl polysaccharide of formula B.

3. An aqueous cleaning composition comprising water and a mixture of alkyl polyglycosides according to claim 1.

4. The aqueous cleaning composition of claim 3, wherein the mixture of alkyl polyglycosides comprise 90 to 100 wt %, based on weight of the aqueous cleaning composition, of alkyl polyglucoside present in the aqueous cleaning composition.

5. The aqueous cleaning composition of claim 4, wherein the mixture of alkyl polyglycosides comprises 5 to 50 wt %, based on weight of the mixture of alkyl polyglycosides, of the branched alkyl polysaccharide of formula B.

6. The aqueous cleaning composition of claim 5, further comprising an ingredient selected from the group consisting of cleaning surfactant other than the mixture of alkyl polyglycosides, a dispersant polymer, a salt, a builder, an enzyme, a corrosion inhibitor, an acid, a base, a bleaching agent, an abrasive, an antimicrobial agent, a chelating agent, a pH adjuster, a buffering agent, a dye, a fragrance, a hydrotrope, an organic solvent, a rheology modifier, sulfonated polymer, poly(alkylene oxide) polymer and mixtures thereof.

7. The aqueous cleaning composition of claim 6, further comprising a cleaning surfactant other than the mixture of alkyl polyglycosides; wherein the cleaning surfactant is selected from an anionic surfactant, a nonionic surfactant other than the mixture of alkyl polyglycosides, a cationic surfactant, a zwitterionic surfactant and mixtures thereof.

8. The aqueous cleaning composition of claim 7, further comprising an organic solvent.

9. The aqueous cleaning composition of claim 8, further comprising a hydrotrope.

10. A method of cleaning a hard surface comprising:
providing a soiled hard surface;
wetting the soiled hard surface with an aqueous cleaning composition according to claim 9; and
wiping the wetted soiled hard surface to provide a cleaned hard surface.
